(19) United States
(12) Patent Application Publication
(10) Pub. No.: US 2025/0262131 A1
(43) Pub. Date: Aug. 21, 2025

(54) METHOD FOR PREPARING A DRY FACIAL MASK AND A DRY FACIAL MASK PREPARED BY THE METHOD

(71) Applicants: NANODOCKS TECHNOLOGY LIMITED, Hong Kong (CN); CITY UNIVERSITY OF HONG KONG, Hong Kong (CN)

(72) Inventors: Jinlian HU, Hong Kong (CN); Yifan SI, Hong Kong (CN); Chunxia GUO, Hong Kong (CN); Siu Wah WONG, Hong Kong (CN)

(21) Appl. No.: 19/016,167

(22) Filed: Jan. 10, 2025

Related U.S. Application Data
(60) Provisional application No. 63/556,099, filed on Feb. 21, 2024.

(51) Int. Cl. A61K 8/02; A61Q 19/00

(52) U.S. Cl. CPC A61K 8/0212; A61Q 19/00; A61K 2800/591; A61K 2800/805

(57) ABSTRACT

The present invention relates to a method for preparing a dry facial mask, comprising the following steps: dissolving a water-soluble polymer into water to form a first solution; adding surfactant into said first solution and then mixing them until dissolved to form a second solution; adding ingredients comprising Chinese medicine into said second solution, and then mixing them until dissolved to form a stock solution; electro-spinning said stock solution onto a substrate to form an intermediate facial mask; and drying said intermediate facial mask to form said dry facial mask.

### FIELD OF THE INVENTION

The present invention generally relates to a preparation method for a facial mask and a facial mask that is prepared by the method. In particular, the present invention focuses on using electrospinning for a warm-sensing Chinese medicine dry facial mask and the dry facial mask prepared by the electrospinning.

### BACKGROUND

There are several types of facial masks available in the market, each designed to target specific skin concerns and provide different benefits. Sheet mask, a type of facial masks in the market, which has gained widespread popularity among consumers due to its convenient use and effective skincare benefits, making it the dominant product in the market. Sheet masks are pre-cut sheets, prepared by loading or infusing typical ingredients such as serum or essence or nutrient or a combination on a substrate usually come in various materials such as cotton, hydrogel, polymer, or bio-cellulose. The typical use of sheet masks is applying the mask on skin for a specific amount of time then removal thereafter.

However, traditional sheet masks have several drawbacks that can negatively impact user experience and effectiveness. One of the issues is inconvenience. These masks often come soaked in liquid serum, which needs to be torn open and applied to the face. This process can be messy, with the serum potentially getting on the user's hands and the package, causing inconvenience. Another problem is the unhygienic usage of traditional masks. Because of the liquid serum, bacteria and pollutants present on the user's hands can contaminate the serum, which then comes into contact with the user's face, creating unsanitary conditions. Additionally, many sheet masks contain organic solvents, which may be used during manufacturing and/or as carriers for the typical ingredients. These solvents are not environmentally friendly and can also be harsh on sensitive skin. Furthermore, when applying the sheet masks, the solvents or liquid ingredients can cause discomfort due to their cooling sensation. Lastly, traditional sheet masks often have a low loading capacity for the typical ingredients, resulting in limited effectiveness in delivering the desired benefits to the skin. These drawbacks highlight the need for innovative solutions that address these issues and offer improved user experience and efficacy.

The increasing demand for a new sheet mask has driven the need for specific properties to address the drawbacks of traditional masks. Firstly, easy manufacturing is desired to streamline production processes. Additionally, it is important to exclude the use of organic solvents during manufacturing to align with environmental protection and minimize potential skin sensitivity issues. The new sheet mask should be dry, eliminating the inconvenience and unhygienic usage associated with liquid-soaked masks. Instead, the typical ingredients should be released upon application of the dry facial mask onto the user's face, ensuring efficient delivery of the desired effects. High loading of these typical ingredients is crucial to enhance the mask's effectiveness. Furthermore, incorporating new ingredients such as Chinese medicine, in particular traditional Chinese medicine, and nutrients into the mask formulation can provide additional cosmetic benefits like wrinkle reduction, skin whitening, and anti-aging effects. Notably, the Chinese medicine may also offer a slightly warming sensation to the user's face, enhancing the overall user experience. By meeting these requirements, the new sheet mask can address the limitations of traditional masks and offer an improved and effective skincare solution.

To cater the demands above, the present invention seeks a method of electrospinning, which is a technique that allows the production of nanofibers having intricate structures and fine fibers, providing high surface area and large surface area-to-volume ratio, thus achieving the following objectives: high loading of ingredients (both typical and new); long retention of nutrients on dry facial mask, and efficient release of ingredients (both typical and new) when the dry facial mask is applied on user's face.

The dry facial mask according to the present invention thus enhances delivery and efficacy in skincare application. Alternatively, the present invention at least provides an alternative to the public.

### SUMMARY OF THE INVENTION

The first aspect of the present invention is related to a method for preparing a dry facial mask, comprising the following steps: i) dissolving a water-soluble polymer into water to form a first solution; ii) adding additives into said first solution and then mixing them until dissolved to form a second solution; iii) adding ingredients comprising Chinese medicine into said second solution, and then mixing them until dissolved to form a stock solution; electro-spinning said stock solution onto a substrate to form an intermediate facial mask; and drying said intermediate facial mask to form said dry facial mask.

In some embodiments, said water-soluble polymer is selected from polyvinyl alcohol PVA, polyethylene oxide, gelatin, or a combination thereof, and the mass ratio of said water-soluble polymer to water is in a range from 6:100 to 10:100.

In some embodiments, said additive is surfactant, the mass ratio of said surfactant to said first solution is in a range from 0.1:100 to 0.3:100.

Preferably, said surfactant is selected from sodium dodecyl sulfate, cetyltrimethylammonium bromide, or a combination thereof.

In some embodiments, the step iii) is characterized by the mass ratio of said Chinese medicine to said second solution is in a range from 2:100 to 5:100.

In some embodiments, in step iii), said ingredients comprising nutrients; and the step iii) is characterized by the mass ratio of said Chinese medicine to said second solution is in a range from 2:100 to 5:100; and the mass ratio of said nutrients to said second solution is in a range from 0.05:100 to 0.2:100.

In some embodiments, said Chinese medicine is selected from vanillin butyl ether, gingerol, cinnamon essential oil, or a combination thereof.

In some embodiments, said Chinese medicine is selected from vanillin butyl ether, gingerol, cinnamon essential oil, or a combination thereof, and said nutrient is selected from niacinamide, gluconolactone, tocopherol acetate, retinol palmitate, panthenol, sodium hyaluronate, or a combination thereof.

Preferably, said substrate is PET non-woven fabric.

More preferably, said substrate is porous super-hydrophilic PET non-woven fabric.

In some embodiments, drying said intermediate facial mask to form said dry facial mask is processed in a heating oven.

Preferably, the thickness of said dry facial mask is 5-15 μm.

Advantageously, said electro spinning is processed with the following parameters:
high pressure of 15-30 kV;
flow rate of injection pump at 0.1-0.4 ml/h;
receiving distance at 5-15 cm; and
spinning time between 120-300 min.

The second aspect of the present invention is related to a dry facial mask, comprising: a substrate preferably prepared by PET non-woven fabric; water-soluble polymer; Chinese medicine, selected from a group comprising vanillin butyl ether, gingerol, cinnamon essential oil, and combination thereof; and optional nutrients, selected from niacinamide, gluconolactone, tocopherol acetate, retinol palmitate, panthenol, sodium hyaluronate, or a combination thereof.

Advantageously, said dry facial mask is prepared by the following steps: i) dissolving said water soluble polymer into water to form a first solution, wherein the mass ratio of said water soluble polymer to water is in a range from 6:100 to 10:100, wherein said water-soluble polymer is selected from polyvinyl alcohol, polyethylene oxide, gelatin, or a combination thereof; ii) adding surfactant into said first solution and then mixing it until dissolved to form a second solution, wherein the mass ratio of said surfactant to said first solution is in a range from 0.1:100 to 0.3:100, and said surfactant is selected from sodium dodecyl sulfate, cetyltrimethylammonium bromide, or a combination thereof; iii) adding said Chinese medicine and said optional nutrients into said second solution, and then mixing it until dissolved to form a stock solution; iv) electro-spinning said stock solution onto said substrate to form an intermediate facial mask v) drying said intermediate facial mask to form said dry facial mask.

Preferably, the step iii) is characterized by: the mass ratio of said Chinese medicine to said second solution is in a range from 2:100 to 5:100; and the mass ratio of said optional nutrients to said second solution is in a range from 0.05:100 to 0.2:100.

Preferably, the step v) is characterized by drying said intermediate facial mask to form said dry facial mask is processed in a heating oven.

Preferably, said electro spinning is processed with the following parameters:
high pressure of 15-30 kV;
flow rate of injection pump at 0.1-0.4 ml/h;
receiving distance at 5-15 cm; and
spinning time between 120-300 min.

Preferably, the thickness of said dry facial mask is 5-15 μm.

### BRIEF DESCRIPTION OF THE DRAWINGS

Some embodiments of the present invention will now be explained, with reference to the accompanied drawings, in which:—

FIG. 1 is a picture showing the electrospinning stock solution of example 1 of the present invention;

FIG. 2 is a picture showing electrospinning stock solution containing additives, Chinese medicine, and nutrients of example 1 of the present invention;

FIG. 3 is a picture showing the dry facial mask of example 1 of the present invention;

FIG. 4A-4B are scanning electron microscope pictures of the dry facial mask of example 2 of the present invention;

FIG. 5 illustrates the use of the dry facial mask of example 3 of the present invention; and

FIGS. 6A-6E illustrate the test results comparing the effects on the skin when a dry facial mask, prepared according to Example 3 of the present invention, is used versus when it is not used.

### DETAILED DESCRIPTION OF PREFERRED EMBODIMENTS OF THE INVENTION

The present invention is now presented by way of examples with reference to the figures in the following paragraphs. Objects, features, and aspects of the present disclosure are disclosed in or are apparent from the following description. It shall be understood by one of ordinary skilled in the art that the following description is a description of exemplary embodiments only, and is not intended as limiting the broader aspects of the present disclosure, which broader aspects are embodied in the exemplary constructions.

It should be noted that, unless otherwise defined, the technical terms or scientific terms used in the embodiments of the present invention shall have the usual meanings understood by person with ordinary skills in the art to which the present invention belongs. “First”, “second” and similar expression used in the embodiments of the present invention do not indicate any order, quantity or importance, but are only used to distinguish different components.

Unless otherwise specified, all chemicals described herein are commercially available and used as received without special handling, and may include impurities, such as residual solvents or by-products. Unless otherwise stated, percentages herein refer to weight percentages. To facilitate the explanation of the present invention, the chemicals used in the description are examples only. It shall be understood that it does not have any limiting effect to the present invention.

The dry facial mask prepared in certain examples below comprise the following general steps:

### Step 1: Preparation of Electrospinning Stock Solution

i) Add a certain mass of water-soluble polymer particles, such as polyvinyl alcohol (PVA), to water. The present invention is not limited with PVA but any other polymer that is suitable for preparing facial mask, such as polyethylene oxide (PEO), gelatin, or a combination thereof. Taking PVA as an example, experimental data shows that mass ratio of PVA to water is in a range from 6:100 to 10:100.
ii) Stir the mixture in i) at a temperature between 70-90° C. for 2-4 hours until the polymer particles are completely dissolved, resulting in an electrospinning stock solution with a specific viscosity and transparent appearance.
iii) The electrospinning stock solution is then reserved at room temperature for further use.

### Step 2: Preparation of Electrospinning Stock Solution Containing Additives, Chinese Medicine, and Nutrients

i) Add certain additives, such as surfactant, to the electrospinning stock solution obtained in Step 1. Experimental data shows that the mass ratio of the surfactant to said the electrospinning stock solution is in a range from 0.1:100 to 0.3:100. Surfactant can be selected from sodium dodecyl sulfate (SDS) or cetyltrimethylammonium bromide (CTAB) or their combination.
ii) Stir the mixture in i) at a temperature between 40-60° C. for 0.5-1 hour until completely dissolved.
iii) Introduce a certain amount of ingredients such as Chinese medicine components into the solution prepared in ii). Experimental data shows that the mass ratio of the Chinese medicine to solution prepared in ii) is in a range from 2:100 to 5:100.
iv) Vigorously stir the mixture in iii) at room temperature for 2 hours, then followed up ultrasonic shock for 0.5-1 hour to obtain a yellow, transparent, and uniform solution.
v) Add a certain amount of ingredients such as nutrients to the solution in iv) and stir it rapidly at 50° C. for 2-4 hours, followed by ultrasonic shock and gentle stirring at room temperature for 0.5-2 hours, resulting in a yellow, uniform, and turbid electrospinning stock solution. Experimental data shows that the mass ratio of the Chinese medicine to solution prepared in ii) is in a range from 0.05:100 to 0.2:100.

### Step 3: Preparation of Electrospun Nanofibers

i) Utilize a single-needle electrospinning device to collect the electrospinning stock solution obtained in Step 2.
ii) Apply high pressure, typically between 15-30 kV, causing the solution prepared in step 2 to be collected on a substrate (e.g., PET non-woven fabric, super-hydrophilic PET, or porous super-hydrophilic PET non-woven fabric) and form a nanofiber film.
iii) Adjust the flow rate of the injection pump to a value between 0.1-0.4 ml/h, set the receiving distance between 5-15 cm, and allow the spinning process to occur for a duration of 120-300 minutes.
iv) After completion, remove the nanofiber film from the roller and place it in a 60° C. oven to dry for 12-48 hours. Experimental data shows that the thickness of the film, i.e., the dry facial mask, is 5-15 μm.

### EXAMPLE 1

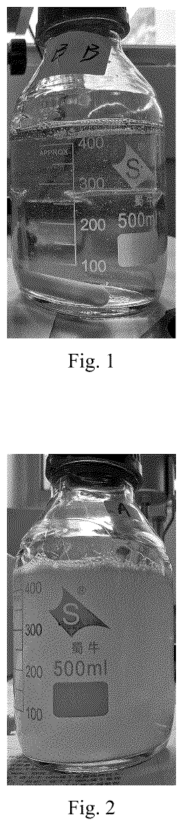

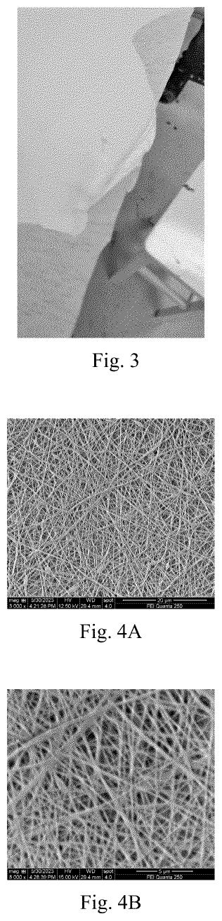

i) 32 grams of PVA is added to 400 gram of water and stirred at 80° C. for 3 hours until completely dissolved. The resulting electrospinning stock solution is viscous and transparent, with certain viscosity and transparent clarification, which is reserved at room temperature, as shown in FIG. 1.
ii) 0.4 gram of sodium dodecyl sulfate is added to the electrospinning stock solution in i) and stir at 40° C. for 0.5 hour until completely dissolved.
iii) Addition of ingredients such as 4 gram of vanillin butyl ether, 4 grams of gingerol, 2 grams of cinnamon essential oil into the solution in ii).
iv) Stir the solution in iii) vigorously at room temperature for 2 hours, and then followed by ultrasonic shock for 0.5 hour to obtain a yellow, transparent and uniform solution.
v) Addition of ingredients such as 0.4 gram of niacinamide, 0.4 gram of gluconolactone, 0.4 gram of tocopherol acetate, 0.4 gram of retinol palmitate, 0.4 gram of panthenol and 0.4 gram of low molecular weight sodium hyaluronate, such as 200-400 kDa, to the solution in iv).
vi) Stir the solution in v) rapidly at 50° C. for 2 hours, then followed by ultrasonic shock for 0.5 hour, and then followed by gentle stirring at room temperature for 1 hour, resulting in a yellow uniform turbid electrospinning solution, as shown in FIG. 2.
vii) Use a single-needle electrospinning device for electrospinning the solution in vi). The electrospinning stock solution is collected on a super-hydrophilic PET substrate under 15 kV high pressure, in which the flow rate of the injection pump is set at 0.2 ml/h, the receiving distance is 15 cm, and the spinning time is 300 minutes.
viii) Remove the electrospun substrate from the roller and dry it in an oven at 60° C. for 24 hours, thus obtaining a nanofiber film containing Chinese medicine and nutrients with thickness about 10 μm, as shown in FIG. 3.

### EXAMPLE 2

i) 36 grams of PVA is added to 400 gram of water and stirred at 90° C. for 2 hours until completely dissolved. The resulting electrospinning stock solution is viscous and transparent, with certain viscosity and transparent clarification, which is reserved at room temperature.
ii) 1.2 gram of sodium dodecyl sulfate is added to the electrospinning stock solution in i) and stir at 60° C. for 0.5 hour until completely dissolved.
iii) Addition of ingredients such as 5 grams of vanillin butyl ether, 5 grams of gingerol into the solution in ii).
iv) Stir the solution in iii) vigorously at room temperature for 2 hours, and then followed by ultrasonic shock for 0.5 hour to obtain a yellow, transparent and uniform solution.
v) Addition of ingredients such as 0.4 gram of niacinamide, 0.4 gram of gluconolactone, and 0.4 gram of low molecular weight sodium hyaluronate to the solution in iv).
vi) Stir the solution in v) rapidly at 50° C. for 4 hours, then followed by ultrasonic shock for 0.5 hour, and then followed by gentle stirring at room temperature for 2 hours, resulting in a yellow uniform turbid electrospinning solution.
vii) Use a single-needle electrospinning device for electrospinning the solution in vi). The electrospinning stock solution is collected on a super-hydrophilic PET substrate under 25 kV high pressure, in which the flow rate of the injection pump is set at 0.1 ml/h, the receiving distance is 10 cm, and the spinning time is 300 minutes.
viii) Remove the electrospun substrate from the roller and dry it in an oven at 60° C. for 12 hours, thus obtaining a nanofiber film containing Chinese medicine and nutrients with thickness about 5 μm. The nanofibers in the film are shown in FIGS. 4A and 4B.

### EXAMPLE 3

i) 40 grams of PVA is added to 400 gram of water and stirred at 70° C. for 4 hours until completely dissolved. The resulting electrospinning stock solution is viscous and transparent, with certain viscosity and transparent clarification, which is reserved at room temperature.
ii) 0.8 gram of sodium dodecyl sulfate is added to the electrospinning stock solution in i) and stir at 40° C. for 0.5 hour until completely dissolved.
iii) Addition of ingredients such as 6 grams of gingerol and 4 grams of cinnamon essential oil into the solution in ii).
iv) Stir the solution in iii) vigorously at room temperature for 2 hours, and then followed by ultrasonic shock for 0.5 hour to obtain a yellow, transparent and uniform solution.
v) Addition of ingredients such as 0.4 gram of tocopherol acetate, 0.4 gram of retinol palmitate, and 0.4 gram of panthenol, and 0.4 gram of low molecular weight sodium hyaluronate to the solution in iv).
vi) Stir the solution in v) rapidly at 50° C. for 2 hours, then followed by ultrasonic shock for 0.5 hour, and then followed by gentle stirring at room temperature for 1 hour, resulting in a yellow uniform turbid electrospinning solution.
vii) Use a single-needle electrospinning device for electrospinning the solution in vi). The electrospinning stock solution is collected on a super-hydrophilic PET substrate under 20 kV high pressure, in which the flow rate of the injection pump is set at 0.4 ml/h, the receiving distance is 15 cm, and the spinning time is 300 minutes.
viii) Remove the electrospun substrate from the roller and dry it in an oven at 60° C. for 24 hours, thus obtaining a nanofiber film containing Chinese medicine and nutrients with thickness about 15 μm.

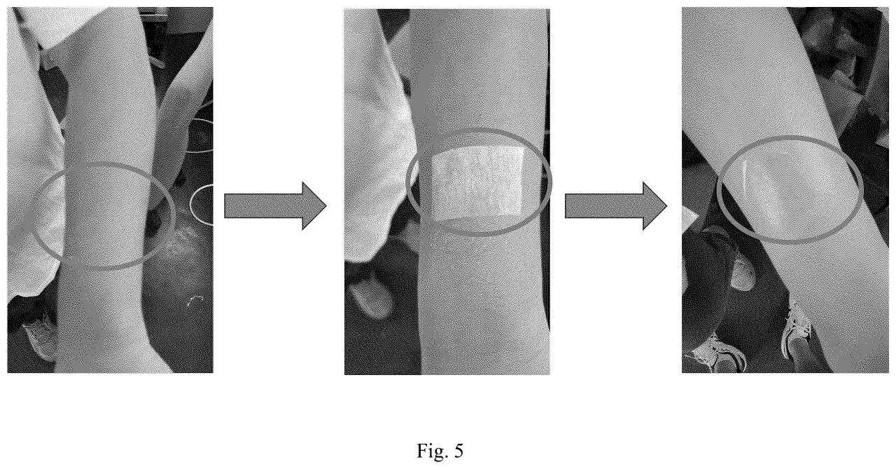

The performance of the nanofiber film containing Chinese medicine and nutrients in Examples 1-3 are tested separately. The test begins by wetting the skin on the arm with a specific quantity of water. Subsequently, a small portion of a warm Chinese medicine nanofiber dry facial mask of each Example, measuring approximately 3 cm×5 cm, is applied to the wet arm. After an approximate duration of 5 minutes, the top layer PET substrate is carefully removed. At this point, it becomes evident that the nanofiber layer, i.e., the PVA nanofibers, has completely dissolved. However, the area of the arm where the mask was initially applied retains its moisture and exhibits a slightly warm sensation. The observed outcome suggests that the warm Chinese medicine nanofiber dry facial mask of the present invention effectively dissolves upon contact with water and successfully retains moisture while providing a gentle warming effect to the skin. FIG. 5 illustrates the use of the dry facial mask of Example 3 of the present invention.

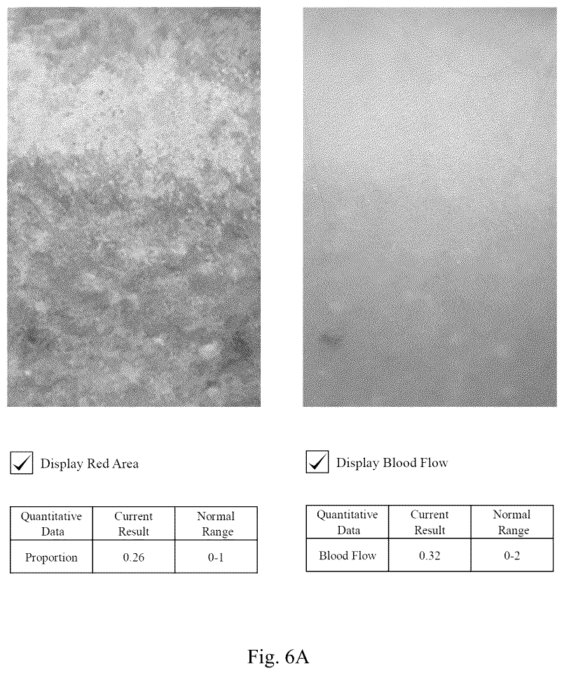

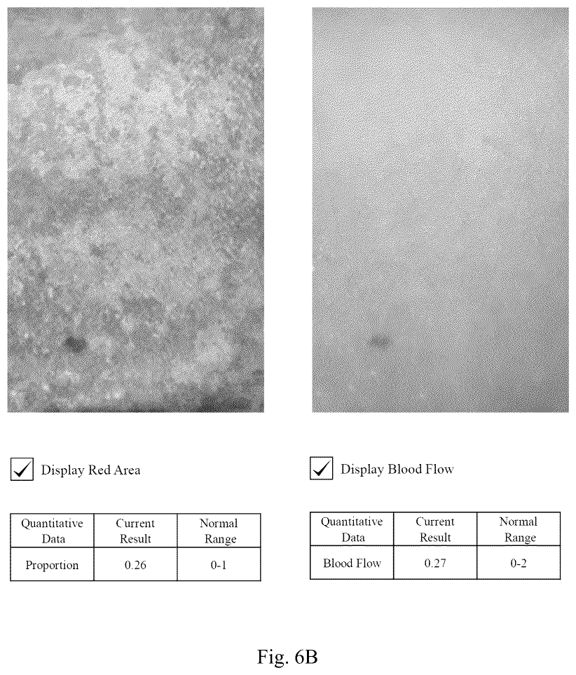

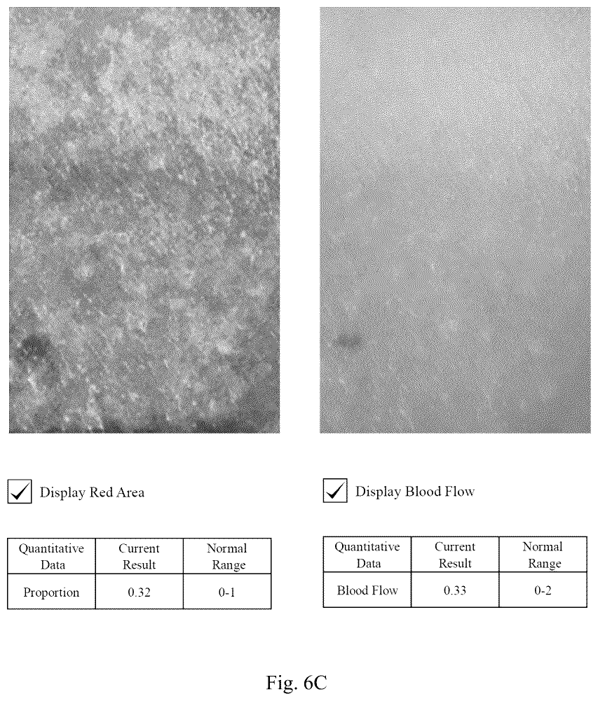

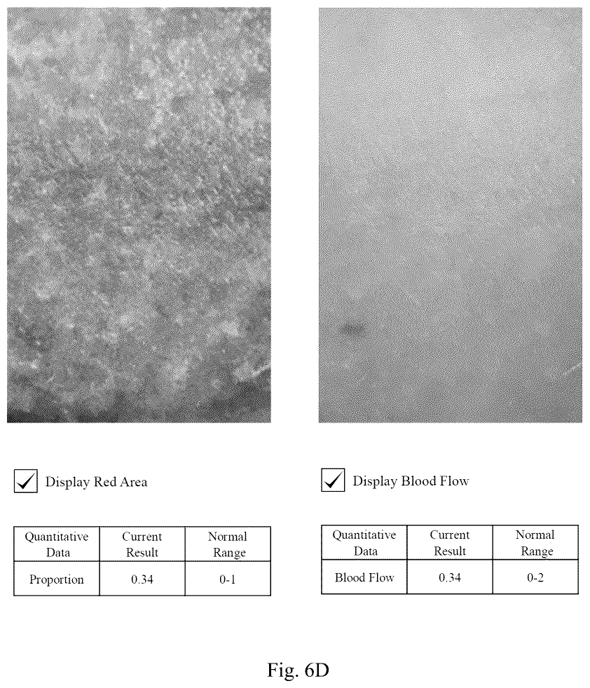

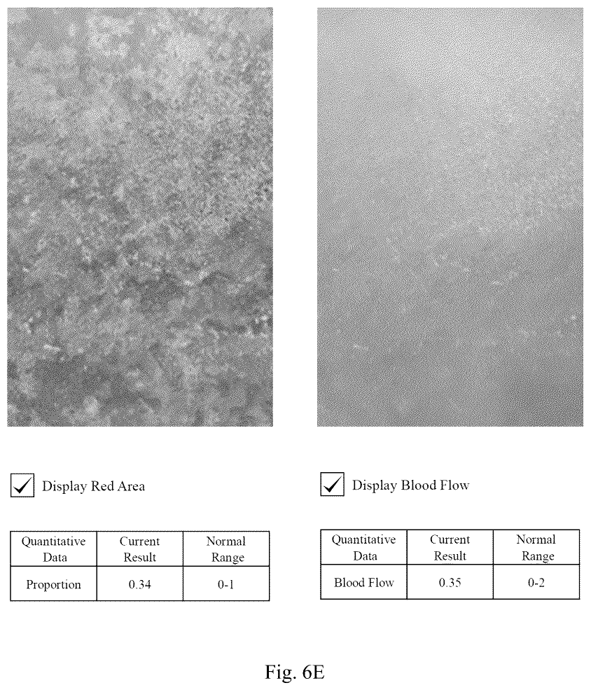

For brevity's sake, the test results comparing the effects on the skin when a dry facial mask, prepared according to Example 3 of the present invention, is used versus when it is not used, are depicted in FIGS. 6A-6E. The purpose of these tests is to evaluate and demonstrate the impact or benefits of using the dry facial mask on the skin. As seen in FIGS. 6A-6E, the red area and blood flow of the skin, being inspected, are visualized as test indicators showing the efficacy of the dry facial masks. FIG. 6A shows the test result of the original skin without using the dry facial mask of Example 3. FIG. 6B shows the test result of the inspected skin immediately after the first use of the dry facial mask of Example 3 for 5 minutes. FIG. 6C shows the test result of the inspected skin 10 minutes after the first use of the dry facial mask of Example 3. FIG. 6D shows the test result of the inspected skin immediately after the second use of the dry facial mask of Example 3 for 5 minutes. The second use was begun 10 minutes after the first use. FIG. 6E shows the test result of the inspected skin 10 minutes after the second use of the dry facial mask of Example 3. The reading of red area refers to the area of red zone over the area of the skin being inspected. The “red area” refers to the measured area of redness on the skin. It is a test indicator used to evaluate the effects of using the dry facial mask. The redness of the skin can be an indication of increased blood flow, and the test measures the extent of this redness in the area being inspected. By comparing the red area before and after using the dry facial mask, the effectiveness of the mask in terms of reducing or enhancing skin redness can be assessed. It shall be understood that the higher reading of the red area and the blood flow, the more active the skin, inferring the higher efficacy the warm sensation provided by the dry facial masks. FIGS. 6A-6E show that upon the first use, the two values did not immediately rise. It can be understood that this may be due to the volatilization of water left on the skin after using the mask. After a ten-minute waiting period, both values showed a significant increase, indicating the effectiveness of the dry facial mask which provides a warm sensation. Upon the second use, the values remained high, further demonstrating the effectiveness of the dry facial mask. It can be seen that after reaching a certain stage, the values of red area and blood flow will not continue to rise. This suggests that the temperature rise from multiple uses does not accumulate and does not pose harm to the human body. If the mask is not used a second time, experimental data shows that the values of the red area and blood flow will decrease gradually.

Aiming at the above technical problems, the invention provides a warm-sensing Chinese medicine, in particular traditional Chinese medicine and nanofiber based, dry facial mask and a preparation method thereof. The masks prepared according to the present invention immediately dissolves and releases ingredients when meeting with water, and exerts its function of whitening and anti-aging. Chinese medicine and nutrients ingredients provide a gentle effect on the skin, and greatly improves user's experience. The present invention at least focuses on two key aspects that set it apart from the state of art. Firstly, the present invention introduces temperature-sensitive Chinese medicine into the realm of dry mask research, imbuing it with temperature-sensitive functionality. This unique feature has not been addressed by the state of art thus far. Secondly, from a technical standpoint, the present invention has successfully achieved the co-solubility of oil-based temperature-sensitive Chinese medicine and water-based polymers by incorporating surfactants. This novel and inventive means has not been previously disclosed by the state of art and its implementation has overcome technical obstacles while providing significant protective value.

The description of the above embodiments is only used to help understanding the method and core idea of the present invention. For those of ordinary skill in the art, without departing from the principle of the present invention, several improvements and modifications can be made to the present invention, and these improvements and modifications also fall within the protection scope of the claims of the present invention. Various modifications to these embodiments are obvious to those skilled in the art, and the general principles defined herein can be implemented in other embodiments without departing from the spirit or scope of the present invention. Therefore, the present invention will not be limited to the embodiments shown in this document but should conform to the widest scope consistent with the principles and novel features disclosed in this document and their equivalents.

1. A method for preparing a dry facial mask, comprising the following steps:
i) dissolving a water-soluble polymer into water to form a first solution;
ii) adding surfactant into said first solution and then mixing them until dissolved to form a second solution;
iii) adding ingredients comprising Chinese medicine into said second solution, and then mixing them until dissolved to form a stock solution;
iv) electro-spinning said stock solution onto a substrate to form an intermediate facial mask; and
v) drying said intermediate facial mask to form said dry facial mask.

2. The method as claimed in claim 1, wherein:—
said water-soluble polymer is selected from polyvinyl alcohol, polyethylene oxide, gelatin, or a combination thereof, and
the mass ratio of said water-soluble polymer to water is in a range from 6:100 to 10:100.

3. The method as claimed in claim 1, wherein the mass ratio of said surfactant to said first solution is in a range from 0.1:100 to 0.3:100.

4. The method as claimed in claim 3, wherein said surfactant is selected from sodium dodecyl sulfate, cetyltrimethylammonium bromide, or a combination thereof.

5. The method as claimed in claim 1, wherein the step iii) is characterized by the mass ratio of said Chinese medicine to said second solution is in a range from 2:100 to 5:100.

6. The method as claimed in claim 1, wherein in step iii), said ingredients comprising nutrients; and the step iii) is characterized by:
the mass ratio of said Chinese medicine to said second solution is in a range from 2:100 to 5:100; and
the mass ratio of said nutrients to said second solution is in a range from 0.05:100 to 0.2:100.

7. The method as claimed in claim 5, wherein said Chinese medicine is selected from vanillin butyl ether, gingerol, cinnamon essential oil, or a combination thereof.

8. The method as claimed in claim 6, wherein:
said Chinese medicine is selected from vanillin butyl ether, gingerol, cinnamon essential oil, or a combination thereof, and
said nutrient is selected from niacinamide, gluconolactone, tocopherol acetate, retinol palmitate, panthenol, sodium hyaluronate, or a combination thereof.

9. The method as claimed in claim 7, wherein said substrate is PET non-woven fabric, preferably said substrate is porous super-hydrophilic PET non-woven fabric.

10. The method as claimed in claim 8, wherein said substrate is PET non-woven fabric, preferably said substrate is porous super-hydrophilic PET non-woven fabric.

11. The method as claimed in claim 9, wherein drying said intermediate facial mask to form said dry facial mask is processed in a heating oven.

12. The method as claimed in claim 10, wherein drying said intermediate facial mask to form said dry facial mask is processed in a heating oven.

13. The method as claimed in claim 11, wherein the thickness of said dry facial mask is 5-15 μm, preferably said electro-spinning is processed with the following parameters:
high pressure of 15-30 kV;
flow rate of injection pump at 0.1-0.4 ml/h;
receiving distance at 5-15 cm; and
spinning time between 120-300 min.

14. The method as claimed in claim 12, wherein the thickness of said dry facial mask is 5-15 μm, preferably said electro-spinning is processed with the following parameters:
high pressure of 15-30 kV;
flow rate of injection pump at 0.1-0.4 ml/h;
receiving distance at 5-15 cm; and
spinning time between 120-300 min.

15. A dry facial mask, comprising:
a substrate;
water-soluble polymer;
Chinese medicine, selected from a group comprising vanillin butyl ether, gingerol, cinnamon essential oil, and combination thereof; and
optional nutrients, selected from niacinamide, gluconolactone, tocopherol acetate, retinol palmitate, panthenol, sodium hyaluronate, or a combination thereof.

16. The dry facial mask according to claim 15, wherein said dry facial mask is prepared by the following steps:
i) dissolving said water soluble polymer into water to form a first solution, wherein the mass ratio of said water soluble polymer to water is in a range from 6:100 to 10:100, wherein said water-soluble polymer is selected from polyvinyl alcohol, polyethylene oxide, gelatin, or a combination thereof;
ii) adding surfactant into said first solution and then mixing it until dissolved to form a second solution, wherein the mass ratio of said surfactant to said first solution is in a range from 0.1:100 to 0.3:100, and said surfactant is selected from sodium dodecyl sulfate, cetyltrimethylammonium bromide, or a combination thereof;
iii) adding said Chinese medicine and said optional nutrients into said second solution, and then mixing it until dissolved to form a stock solution;
iv) electro-spinning said stock solution onto said substrate to form an intermediate facial mask
v) drying said intermediate facial mask to form said dry facial mask.

17. The dry facial mask according to claim 16, wherein the step iii) is characterized by:
the mass ratio of said Chinese medicine to said second solution is in a range from 2:100 to 5:100; and
the mass ratio of said optional nutrients to said second solution is in a range from 0.05:100 to 0.2:100.

18. The dry facial mask according to claim 17, wherein the step v) is characterized by drying said intermediate facial mask to form said dry facial mask is processed in a heating oven.

19. The dry facial mask according to claim 18, wherein said electro-spinning is processed with the following parameters:
high pressure of 15-30 kV;
flow rate of injection pump at 0.1-0.4 ml/h;
receiving distance at 5-15 cm; and
spinning time between 120-300 min.

20. The dry facial mask according to claim 19, wherein the thickness of said dry facial mask is 5-15 μm.
